(19) United States
(12) Patent Application Publication
(10) Pub. No.: US 2013/0123204 A1
(43) Pub. Date: May 16, 2013

(54) METHOD FOR TREATING HEPATITIS B

(71) Applicants: Lain-Tze LEE, Hsinchu City (TW); Shau-Feng CHANG, Hsinchu City (TW); Chia-Yu LIN, Hsinchu City (TW); Hui-Yen WU, Hsinchu County (TW); Shu-Feng CHEN, Changhua County (TW); Jinun-Ban YEH, Hsinchu City (TW)

(72) Inventors: Lain-Tze LEE, Hsinchu City (TW); Shau-Feng CHANG, Hsinchu City (TW); Chia-Yu LIN, Hsinchu City (TW); Hui-Yen WU, Hsinchu County (TW); Shu-Feng CHEN, Changhua County (TW); Jinun-Ban YEH, Hsinchu City (TW)

(73) Assignee: INDUSTRIAL TECHNOLOGY RESEARCH INSTITUTE, Hsinchu (TW)

(21) Appl. No.: 13/735,620

(22) Filed: Jan. 7, 2013

Related U.S. Application Data
(63) Continuation-in-part of application No. 12/648,986, filed on Dec. 29, 2009.

(30) Foreign Application Priority Data
Dec. 31, 2008 (TW) 097151799

(51) Int. Cl. A61K 31/352

(52) U.S. Cl. CPC A61K 31/352
USPC 514/27; 514/456

(57) ABSTRACT

The disclosure provides a method for treating hepatitis B, including: administering an effective amount of proanthocyanidins wherein the formula of a monomer of the proanthocyanidins is shown as Formula (I):

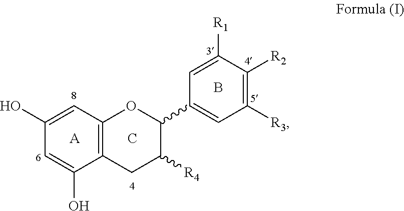

wherein R₁ is OCH₃, R₂ is OH and R₃ is H, or R₁ is OH and R₂ and R₃ both are H, or R₁ and R₂ both are OH and R₃ is H, or R₁, R₂ and R₃ all are OH, and R₄ is 3-(α)-OH, 3-(β)-OH, 3-(α)-O-sugar or 3-(β)-O-sugar. Bonding between adjacent monomers take place at C4, C8 Carbon-carbon bond or C2, C7 oxygen bond of A type or C4, C8 or C4, C6 Carbon-carbon bond of B type or C type of proanthocyanidins. And the polymerized number is from 2 to 30.

### CROSS REFERENCE TO RELATED APPLICATIONS

This application is a Continuation-In-Part of pending U.S. patent application Ser. No. 12/648,986, filed Dec. 29, 2009 and entitled “Plant extract composition for treating hepatitis C” which claims priority to Taiwan Patent Application No. 097151799, filed Dec. 31, 2008, both of which are disclosed herein in their entirety.

### TECHNICAL FIELD

The present disclosure relates to a method for treating hepatitis B, and in particular relates to a method for treating hepatitis B through administering a proanthocyanidins which is able to inhibit hepatitis B virus activity.

### BACKGROUND

Presently, there are 0.35 billion asymptomatic hepatitis B virus carriers in the word population that are known, and 300 thousand people die annually, due to hepatitis B associated diseases. In Taiwan, excess 8 thousand people die annually, due to hepatitis B associated diseases. Currently, there is no effective cure for hepatitis B. However, while anti-hepatitis B drugs such as Interferon and Lamivudine have been disclosed, they induce serious side effects in patients.

Therefore, there is a need for anti-hepatitis B drug compositions that do not induce serious side effects in patients. As such, anti-hepatitis B drug compositions including natural plants have been proposed.

It has been disclosed that the natural herb Boehmeria nivea (L.) Gaud has administrative effects such as liver function enhancement and anti-inflammation, without serious side effects in patients. However, using Boehmeria nivea (L.) Gaud in the treatment of hepatitis B has yet to be disclosed and identifying active ingredients thereof, have yet to be conclusively determined

Meanwhile, proanthocyanidins extracted from natural plants has excellent anti-oxidation effects and is usually used as a health food product. Taiwan Patent Number I274551 discloses a nutriment containing taurine, β-Carotene, proanthocyanidins extracted from grape seeds, vitamin E and vitamin C. The nutriment has been found to have an effect on improving chronic hepatitis. However, whether proanthocyanidins is able to inhibit hepatitis B effectively has yet to be determined.

### BRIEF SUMMARY

The disclosure provides a pharmaceutical composition for treating hepatitis B, comprising an effective amount of proanthocyanidins, wherein a formula of a monomer of the proanthocyanidins is shown as Formula (I):

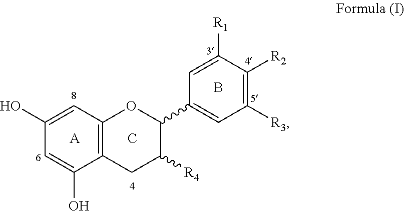

wherein R₁ is OCH₃, R₂ is OH and R₃ is H, or R₁ is OH and R₂ and R₃ both are H, or R₁ and R₂ both are OH and R₃ is H, or R₁, R₂ and R₃ all are OH, and R₄ is 3-(α)-OH, 3-(β)-OH, 3-(α)-O-sugar or 3-(β)-O-sugar; and a pharmaceutically acceptable carrier or salt. A bonding between adjacent monomers of the proanthocyanidins take place at C4, C8 carbon-carbon bond, C4, C6 carbon-carbon bond or C2, C7 oxygen bond and the isomer thereof and, the polymerized number of the proanthocyanidins is from 2 to 30.

The disclosure also provides a health food product having hepatitis B virus inhibiting effects, comprising an effective amount of proanthocyanidins, wherein a formula of a monomer of the proanthocyanidins is shown as Formula (I):

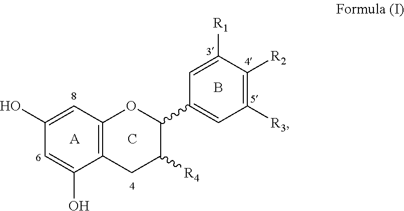

wherein R₁ is OCH₃, R₂ is OH and R₃ is H, or R₁ is OH and R₂ and R₃ both are H, or R₁ and R₂ both are OH and R₃ is H, or R₁, R₂ and R₃ all are OH, and R₄ is 3-(α)-OH, 3-(β)-OH, 3-(α)-O-sugar or 3-(β)-O-sugar; and a pharmaceutically acceptable carrier or salt. A bonding between adjacent monomers of the proanthocyanidins take place at C4, C8 carbon-carbon bond, C4, C6 carbon-carbon bond or C2, C7 oxygen bond and the isomer thereof and, the polymerized number of the proanthocyanidins is from 2 to 30.

The disclosure further provides a method for treating hepatitis B, comprising: administering an effective amount of proanthocyanidins wherein the formula of a monomer of the proanthocyanidins is shown as Formula (I):

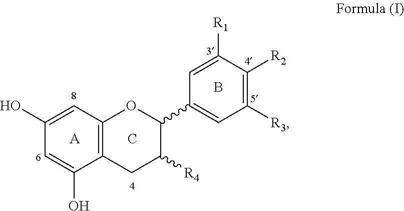

wherein R₁ is OCH₃, R₂ is OH and R₃ is H, or R₁ is OH and R₂ and R₃ both are H, or R₁ and R₂ both are OH and R₃ is H, or R₁, R₂ and R₃ all are OH, and R₄ is 3-(α)-OH, 3-(β)-OH, 3-(α)-O-sugar or 3-(β)-O-sugar.

A detailed description is given in the following embodiments with reference to the accompanying drawings.

### BRIEF DESCRIPTION OF THE DRAWINGS

The embodiments of disclosure can be more fully understood by reading the subsequent detailed description and examples with references made to the accompanying drawings, wherein:

FIGS. 1a-1f show the formulas of 3-flavanol, 3,4-flavanol, catechin ((2R,3S) and (2S,3R)) and epicatechin ((2S,3S) and (2R,3R)), respectively;

FIG. 2a and FIGS. 2b-e show the evolved gas analysis-mass spectrum and structure analysis of the proanthocyanidins, respectively;

FIG. 3 shows the infrared spectroscopy analysis for the purified proanthocyanidins sample;

FIGS. 4a-4b shows a high-performance liquid chromatography-ESL⁺ mass spectrum and the enlarged mass spectrum of dimmer and trimer for the purified proanthocyanidins;

FIGS. 5a-5c show the ¹³C magnetic resonance spectroscopy results;

FIGS. 6a and 6b show the bounding between two monomers of the proanthocyanidins;

FIG. 7a-c show the matrix assisted laser desorption ionization time-of-flight (MALDI-TOF) mass spectra of the partial purified proanthocyanidins;

FIGS. 8a and 8b show inhibiting hepatitis B virus activity analysis for the proanthocyanidins extract from the plants belonging to different families; and

FIGS. 9a and 9b show inhibiting hepatitis B virus activity analysis for the proanthocyanidins.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The disclosure uses the proanthocyanidins as an effective ingredient of a pharmaceutical composition or a health food for treating hepatitis B (and/or hepatitis B associated diseases) and for inhibiting hepatitis B virus activity.

The proanthocyanidins having hepatitis B virus inhibiting effects may be extracted from a plant. In one embodiment, the plant may comprise a plant belonging to the Leguminosae, Crassulaceae, Combretaceae, Asclepiadaceae, Rosaceae, Lamiaceae, Polygonaceae, Ericaceae, Pinaceae, Vitaceae or Urticaceae family, and preferably is Boehmeria nivea (L.) Gaud belonging to the Urticaceae family. The part of the plant to be extracted may comprise a root, a stem, a leaf and/or a fruit part.

Moreover, in the present disclosure, the plant extracting process may be performed by a well known method. In one embodiment, after drying, the root, stem, leaf and/or fruit of the plant is/are cut or grinded and then is extracted by an extraction solution. In one embodiment, the roots and/or stems of Boehmeria nivea (L.) Gaud is extracted.

Water or a mixture solution of water and a solvent with different polarity may be selected as the extraction solution. The solvent with different polarity may comprise ethanol, acetone, methanol or ethyl acetate. These solvents may be used alone or mixed together or mixed with water. A ratio between the extraction solution and the plant is not be limited, and in one embodiment, the ratio between the extraction solution and the plant is 1:10 (W/V).

Furthermore, the extraction temperature may change with the extraction solution which is used. In one embodiment, the extraction process may be performed in the extraction solution at room temperature. In other embodiments, the extraction process may be heated to a different reflux temperature according to the extraction solution which is used. The extraction time may be between 2 hours and 7 days according the extraction temperature. During extraction, sodium chloride, diluted inorganic acid (such as diluted hydrochloric acid) or organic acid (such as vitamin C, tartaric acid) may be added into the extraction solution optionally to adjust the pH value of the extraction solution.

Then, an extract with the proanthocyanidins as an active ingredient is dried following concentration. Alternatively, the extract may be further partially purified or completely purified, optionally. In one embodiment, processes of the partial purification comprises the dried extract being redissolved in a 95% ethanol and/or methanol aqueous solution, and then extracted by a solvent with different polarity from the 95% ethanol and/or methanol aqueous solution to remove impurities. For example, the lipid and non polar substance of the extract is removed by a non-polar solvent (such as hexane) and then the extract is extracted by chloroform and/or ethyl acetate to remove the phenolics compounds with low molecular weight. After that, the water layer of the extract having been extracted by the solvents mentioned above is concentrated and dried to obtain the partial purified proanthocyanidins.

Processes of complete purification may comprise the partial purified extract being dissolved in an ethanol or methanol aqueous solution, placed into a gel permeation chromatography column, and then eluted with different solutions and/or a mixed solution to perform the purification and isolation of the proanthocyanidins. In one embodiment, the different solutions used for elution, include 95% ethanol, 95% ethanol/methanol (1:1, v/v),100% methanol, 50% methanol and 50% acetone aqueous solutions, 100% acetone in order. The solutions eluted out by the different solutions are fractionally collected, respectively, and then detected the proanthocyanidins therein with a 280 nm absorption value as detected by liquid chromatography. The solutions having the proanthocyanidins with different molecular weight distributions, respectively may be obtained by collecting the solutions eluted out by the different solutions. The solutions eluted out by the different solutions and fractionally collected are concentrated at a temperature lower than 40 and lyophilized to obtain the purified proanthocyanidins. In one embodiment, the gel permeation chromatography column may be a Sephadex LH-20 column (GE Pharmacia).

A formula of a monomer of the proanthocyanidins purified by the processes mentioned above is shown as Formula (I):

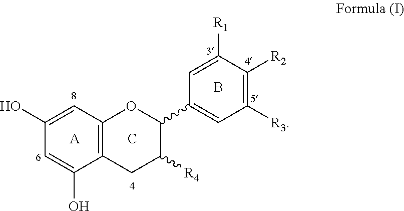

In one embodiment, R₁ is OCH₃, R₂ is OH and R₃ is H. In other embodiments, R₁ is OH and R₂ and R₃ both are H. Or R₁ and R₂ both are OH and R₃ is H. Alternatively, R₁, R₂ and R₃ all are OH. Moreover, R₄ may be 3-(α)-OH, 3-(β)-OH, 3-(α)-O-sugar or 3-(β)-O-sugar.

Moreover, the monomer may comprise (R) or (S) optical isomers at C4.

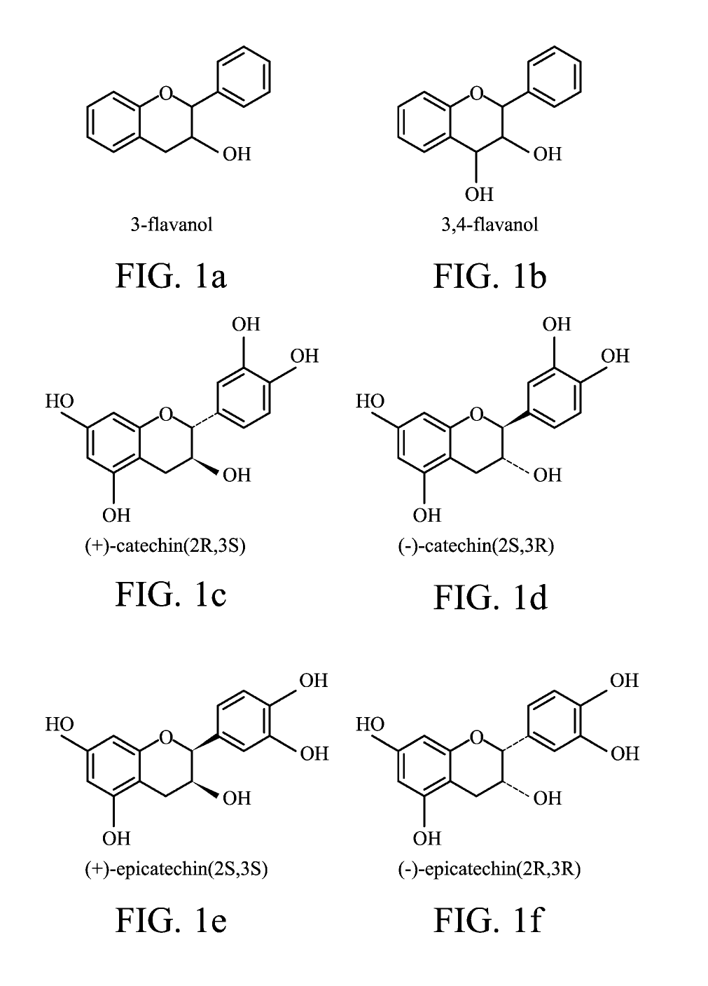

Furthermore, the monomer may comprise a flavonoid. The flavonoid may comprise catechin, epicatechin, epiafzelechin, gallocatechin, galloepicatechin, epigallocatechin, gallates, flavonols, flavandiols, leucocyanidins or procynidins. In one embodiment, the monomer of the proanthocyanidins may comprise flavan-3-ol or flavanones derivatives. The formulas of 3-flavanol, 3,4-flavanol, catechin ((2R,3S) and (2S,3R)) and epicatechin ((2S,3S) and (2R,3R)) are shown in FIGS. 1a-1f, respectively.

A polymerized number of the purified proanthocyanidins oligomer may be about 2-30, preferably, 3-20. A bonding between adjacent monomers of the proanthocyanidins oligomer may take place at C4, C8 carbon-carbon bond, C4, C6 carbon-carbon bond or C2, C7 oxygen bond. The average molecular weight of the proanthocyanidins may be about 600-10000.

In one embodiment, the purified proanthocyanidins comprise proanthocyanidins with low oligomers; dimer, trimer and tetramer. In other embodiments, the purified proanthocyanidins comprise a mixture of proanthocyanidins with different degrees of polymerization.

In one embodiment, in the formula of the monomer of the proanthocyanidins, R₁ is OCH₃, R₂ is OH and R₃ is H or R₁ is OH and R₂ and R₃ both are H or R₁ and R₂ both are OH and R₃ is H or R₁, R₂ and R₃ all are OH. The 50% inhibition concentration (IC₅₀) of the proanthocyanidins having the monomer with different R₁s for hepatitis B virus surface antigen is about 127-164 μg/ml and the 50% inhibition concentration (IC₅₀) of the proanthocyanidins having the monomer with different R₁s for hepatitis B virus e antigen is about 44-84 μg/ml. Thus, different R₁ of the monomer do not influence inhibition of hepatitis B virus bioactivity of the proanthocyanidins.

In the present disclosure, the extracted proanthocyanidins mentioned above may be formed as a pharmaceutical composition for treating hepatitis B comprising the proanthocyanidins and a pharmaceutically acceptable carrier or salt.

The pharmaceutically acceptable carrier may comprise, but is not limited to, a solvent, a dispersion medium, a coating, an antibacterial and antifungal agent, or an isotonic and absorption delaying agent. The pharmaceutical composition can be formulated into dosage forms for different administration routes utilizing conventional methods.

The pharmaceutically acceptable salt may comprise, but is not limited to, inorganic cation salt, such as alkali metal salts such as sodium salt, potassium salt or amine salt, such as alkaline-earth metal salt such as magnesium salt or calcium salt, such as the salt containing bivalent or quadrivalent cation such as zinc salt, aluminum salt or zirconium salt. In addition, the pharmaceutically acceptable salt may also comprise organic salt, such as dicyclohexylamine salt, methyl-D-glucamine, and amino acid salt such as arginine, lysine, histidine, or glutamine.

The pharmaceutical composition may be administered orally, parenterally by an inhalation spray or via an implanted reservoir. The parenteral method may comprise subcutaneous, intracutaneous, intravenous, intramuscular, intraarticular, intraarterial, intrasynovial, intrasternal, intrathecal, and intraleaional, as well as infusion techniques.

An oral composition can comprise, but is not limited to, tablets, capsules, emulsions and aqueous suspensions, dispersions and solutions.

Alternatively, the present disclosure also provides a health food which comprises the proanthocyanidins mentioned above, and thus the health food product has hepatitis B virus inhibiting effects.

The disclosure further provides a method for treating hepatitis B. The method for treating hepatitis B may comprise, but is not limited to administering an effective amount of proanthocyanidins wherein the formula of a monomer of the proanthocyanidins is shown as Formula (I):

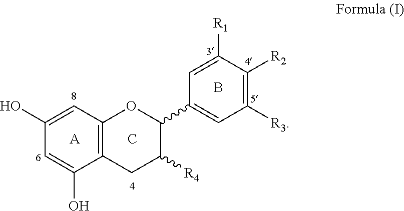

The proanthocyanidins mentioned above have hepatitis B virus inhibiting effects.

In one embodiment, R₁ is OCH₃, R₂ is OH and R₃ is H. In other embodiments, R₁ is OH and R₂ and R₃ both are H. Or R₁ and R₂ both are OH and R₃ is H. Alternatively, R₁, R₂ and R₃ all are OH. Moreover, R₄ may be 3-(α)-OH, 3-(β)-OH, 3-(α)-O-sugar or 3-(β)-O-sugar.

In addition, the monomer may comprise (R) or (S) optical isomers at C4.

Furthermore, the monomer may comprise a flavonoid. The flavonoid may comprise catechin, epicatechin, epiafzelechin, gallocatechin, galloepicatechin, epigallocatechin, flavonols, flavandiols, leucocyanidins or procynidins. In one embodiment, the monomer of the proanthocyanidins may comprise flavan-3-ol or flavanones derivatives. The formulas of 3-flavanol, 3,4-flavanol, catechin ((2R,3S) and (2S,3R)) and epicatechin ((2S,3S) and (2R,3R)) are shown in FIGS. 1a-1f, respectively.

A polymerized number of the purified proanthocyanidins oligomer may be about 2-30, preferably, 3-20. A bonding between adjacent monomers of the proanthocyanidins oligomer may take place at C4, C8 carbon-carbon bond, C4, C6 carbon-carbon bond or C2, C7 oxygen bond. The average molecular weight of the proanthocyanidins may be about 600-10000.

In one embodiment, the purified proanthocyanidins comprise proanthocyanidins with low oligomers; dimer, trimer and tetramer. In other embodiments, the purified proanthocyanidins comprise a mixture of proanthocyanidins with different degrees of polymerization.

In one embodiment, in the formula of the monomer of the proanthocyanidins, R₁ is OCH₃, R₂ is OH and R₃ is H or R₁ is OH and R₂ and R₃ both are H or R₁ and R₂ both are OH and R₃ is H or R₁, R₂ and R₃ all are OH. The 50% inhibition concentration (IC₅₀) of the proanthocyanidins having the monomer with different R1s for hepatitis B virus surface antigen is about 127-164 μg/ml and the 50% inhibition concentration (IC₅₀) of the proanthocyanidins having the monomer with different R₁s for hepatitis B virus e antigen is about 44-84 μg/ml. Thus, different R1 of the monomer do not influence inhibition of hepatitis B virus bioactivity of the proanthocyanidins.

The proanthocyanidins having hepatitis B virus inhibiting effects may be extracted from a plant. In one embodiment, the plant may comprise a plant belonging to the Leguminosae, Crassulaceae, Combretaceae, Asclepiadaceae, Rosaceae, Lamiaceae, Polygonaceae, Ericaceae, Pinaceae, Vitaceae or Urticaceae family, and preferably is Boehmeria nivea (L.) Gaud belonging to the Urticaceae family. The part of the plant to be extracted may comprise a root, a stem, a leaf and/or a fruit part.

Moreover, in the present disclosure, the plant extracting process may be performed by a well known method. In one embodiment, after drying, the root, stem, leaf and/or fruit of the plant is/are cut or grinded and then is extracted by an extraction solution. In one embodiment, the roots and/or stems of Boehmeria nivea (L.) Gaud is extracted.

### EXAMPLE

### Example 1

Determining the Structure of the Monomer of the Proanthocyanidins

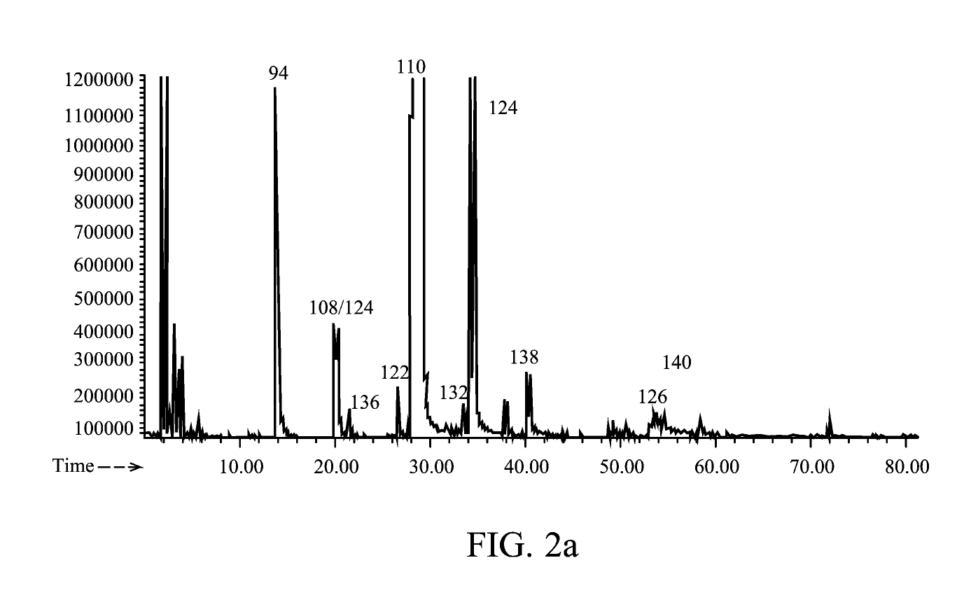

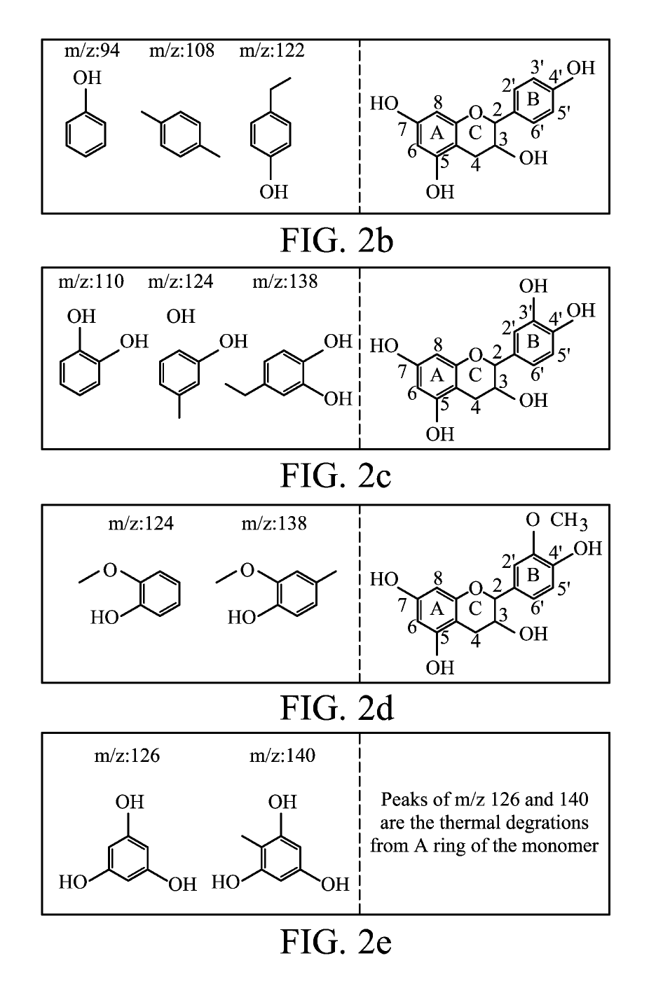

The structure of the monomer of the proanthocyanidins was determined by evolved gas analysis-mass spectrometry (EGA/MS). Solid and purified proanthocyanidins were placed into the evolved gas analysis-mass spectrometry to be progressively heated at stage temperatures (50-500° C.) or heated_at a suitable single shot temperature. The heat decomposed sample was separated by the specific metal column of the evolved gas analysis-mass spectrometry and then detected by a mass spectrometry detector to generate a mass spectrum. According the mass spectra, the structure of the monomer of the proanthocyanidins was determined The pyrogram and structure of thermal degradation peaks from the proanthocyanidins are shown in FIG. 2a and FIGS. 2b-e, respectively. The left parts of FIGS. 2b-e shows the m/z values and chemical structures of the peaks for thermal degradations from B ring in FIG. 2a, and the right parts of FIGS. 2b-e show the monomer analysis results for the chemical structures of the peaks in the left parts, respectively. Peaks of m/z 126 and 140 are the thermal degradations from A ring and the other m/z peaks are the thermal degradations from B ring of the proanthocyanidins monomers. The determined formula of the monomer of the proanthocyanidins is shown as Formula (I):

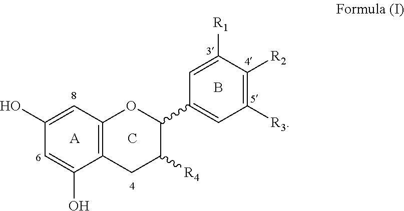

R₁ is OCH₃, R₂ is OH and R₃ is H, or R₁ is OH and R₂ and R₃ both are H, or R₁ and R₂ both are OH and R₃ is H, or R₁, R₂ and R₃ all are OH.

Moreover, the mass spectrum also shows the peaks for glucosides, and thus the composition of R4 may be 3-(α)-OH, 3-(β)-OH, 3-(α)-O-sugar or 3-(β)-O-sugar.

### Example 2

Preparation of the Boehmeria nivea (L.) Gaud Extract Having the Proanthocyanidins

Method 1

The roots and stems of Boehmeria nivea (L.) Gaud were washed and dried in a natural environment. The dried Boehmeria nivea (L.) Gaud was cut into 5 mm thick slices and stored at 4. Then the stored Boehmeria nivea (L.) Gaud was grinded by a grinder and then screened. The screened power less than 20 mesh was taken and added into 95% ethanol (1:10, w/v), heated and refluxed for 2 hours (performed twice) and then cooled to room temperature. The heated and then cooled to room temperature extract solution was put into a centrifuge bag to be filtered by centrifuging. The filtered solution was concentrated by a vacuum evaporator at a temperature lower than 40, and then lyophilized by a lyophilizer The lyophilized extract was a pharmaceutical composition containing an ingredient of proanthocyanidins and had hepatitis B virus inhibiting activities.

Method 2

The Boehmeria nivea (L.) Gaud stored at 4 in method 1 was grinded by a grinder and then screened. The screened power less than 20 mesh was taken and added into RO water (1:10, w/v), heated and refluxed for 2 hours (performed twice) and then cooled to room temperature. The heated and then cooled to room temperature extract solution was added into an ethanol aqueous solution (95-50%) and mixed. After the extract solution was cooled and precipitated, the upper layer solution was added into a centrifuge bag to be filtered by centrifuging. The filtered solution was concentrated by a vacuum evaporator at a temperature lower than 40, and then lyophilized by a lyophilizer. The lyophilized extract was a pharmaceutical composition containing an ingredient of proanthocyanidins and had hepatitis B virus inhibiting activities.

### Example 3

Partial Purification of the Boehmeria nivea (L.) Gaud Extract

Method 1

Solvent Extracting-1

The Boehmeria nivea (L.) Gaud extract containing proanthocyanidins was added into a hexane (1:10 w/v), heated and refluxed for 6 hours to remove the lipid in the extract. The solid extract was dissolved in 70% methanol aqueous solution and/or 0.3% vitamin C solution and concentrated by a vacuum evaporator at a temperature lower than to remove the solvent. Then, the extract was added into chloroform (extract: chloroform=1:1, v/v) and vortexed for 30 minutes (multiple extractions were performed). The water layer therefrom was added into ethyl acetate (extract:ethyl acetate=1:1, v/v) and vortexed for 30 minutes (multiple extractions were performed). The water layer therefrom was concentrated by a vacuum evaporator at a temperature lower than 40° C., and then lyophilized by a lyophilizer.

Method 2

Solvent Extracting-2

The Boehmeria nivea (L.) Gaud extract containing proanthocyanidins was dissolved in a water/ethanol solution removed ethanol by a vacuum evaporator at a temperature lower than 40° C., added into a hexane (1:10 v/v) and then vortexed for 30 minutes (multiple extractions were performed) to remove the lipid in the extract. The water layer therefrom was added into ethyl acetate (water layer:ethyl acetate=1:1, v/v) and vortexed for 30 minutes (multiple extractions were performed). The water layer therefrom was added into 1-butanol (1:1, v/v) and vortexed for 30 minutes (multiple extractions were performed). The water layer therefrom was concentrated by a vacuum evaporator at a temperature lower than 40° C., and then lyophilized by a lyophilizer

Method 3

Gel Permeation Chromatography

The partial purified Boehmeria nivea (L.) Gaud extract containing proanthocyanidins in the method 1 was isolated by gel permeation chromatography (4 cm diameter×45 cm long Sephadex LH-20) by using the solutions having different polarity ratios to elute, and remove impurities therein. 2.5 g of the partial purified substance was dissolved in 0.5 ml of 95% ethanol and placed into the gel permeation chromatography column and then continuously eluted with a serial of solvents. The eluted solutions eluted by different solvents were collected. The solvents were 300 ml of 95% ethanol, 300 ml of 95% ethanol/methanol (1/1. v/v), 300 ml of methanol, 300 ml of 50% methanol aqueous solution and 300 ml of 50% acetone aqueous solution, 300 ml of acetone respectively. Except for the eluted solution eluted by 300 ml of 95% ethanol, all other eluted solutions were concentrated by a vacuum evaporator at a temperature lower than 40° C., and then lyophilized by a lyophilizer The lyophilized substance was stored at −20° C. for ready for use. The physical and chemical properties of the lyophilized Boehmeria nivea (L.) Gaud extract with partially purified and/or purified proanthocyanidins was analyzed. The lyophilized eluted substance had a partially purified and/or purified proanthocyanidins ingredient and was a drug having hepatitis B virus inhibiting effects.

### Example 4

Infrared Spectroscopy Analysis

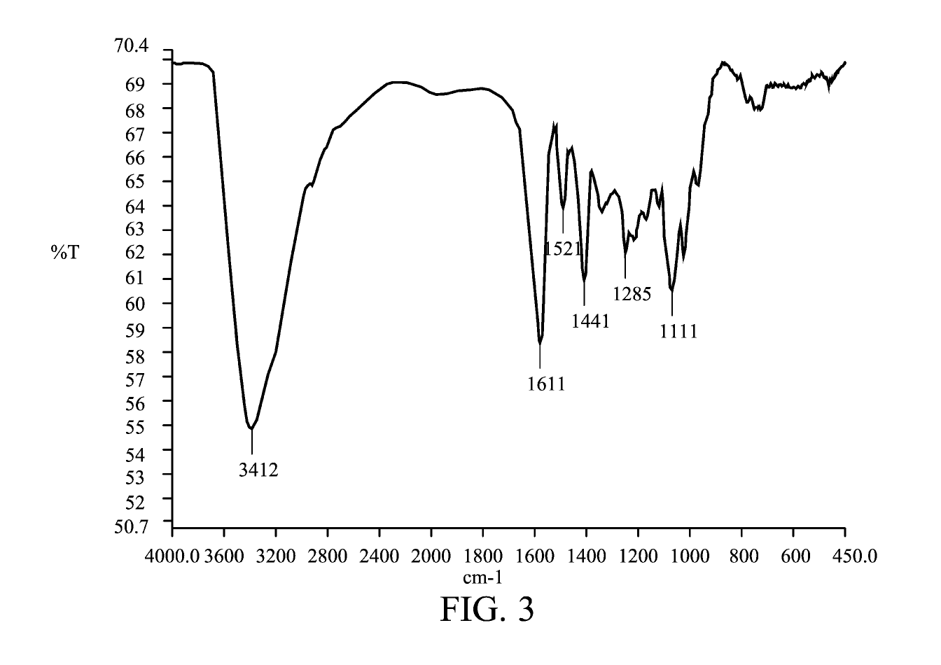

The purified proanthocyanidins sample was mixed with potassium chloride, pressed to be a film and then detected by infrared spectroscopy. The results are shown in FIG. 3, wherein the stronger absorbing peaks are 3412.38 nm, 1611 nm, 15211 nm, 1441.14 nm, 1285 nm and 1111 nm.

### Example 5

High-Performance Liquid Chromatography-Mass Spectroscopy Analysis

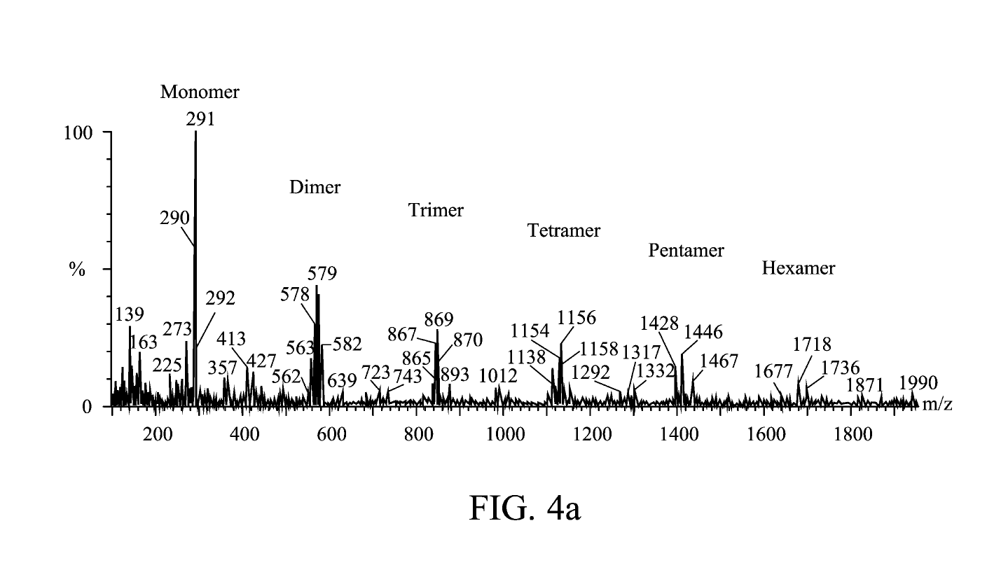

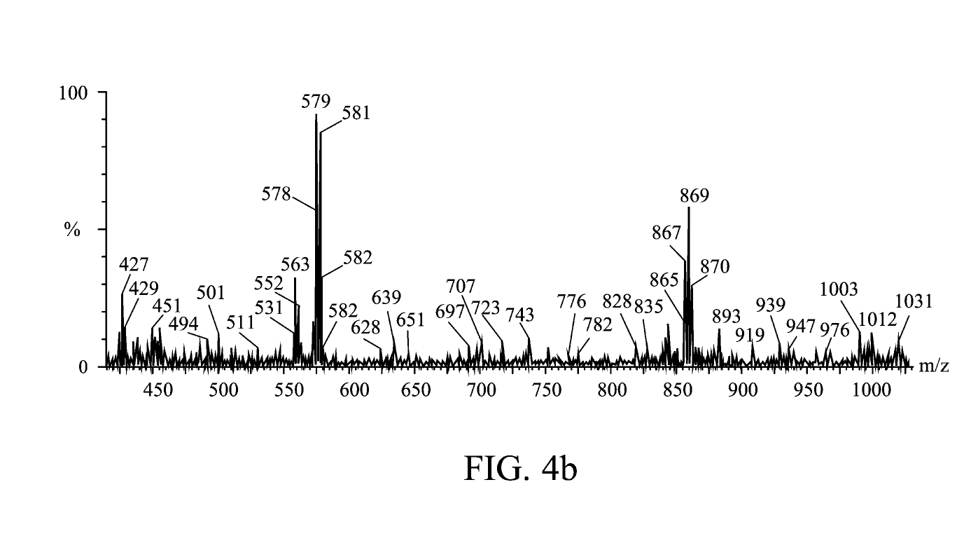

The purified proanthocyanidins sample was analyzed by high-performance liquid chromatography-mass spectrometry (HPLC/ESI⁺) (Micromass Quattro/Waters 2690). The monomer and the polymer of the proanthocyanidins with 1-6 polymerization degrees and a proanthocyanidins monomer with a glucoside having a molecular weight of 164 were detected. The high-performance liquid chromatography-ESI mass spectrum for purified proanthocyanidin is shown in FIG. 4a.

### Example 6

¹³C and ¹H Nuclear Magnetic Resonance Spectroscopy

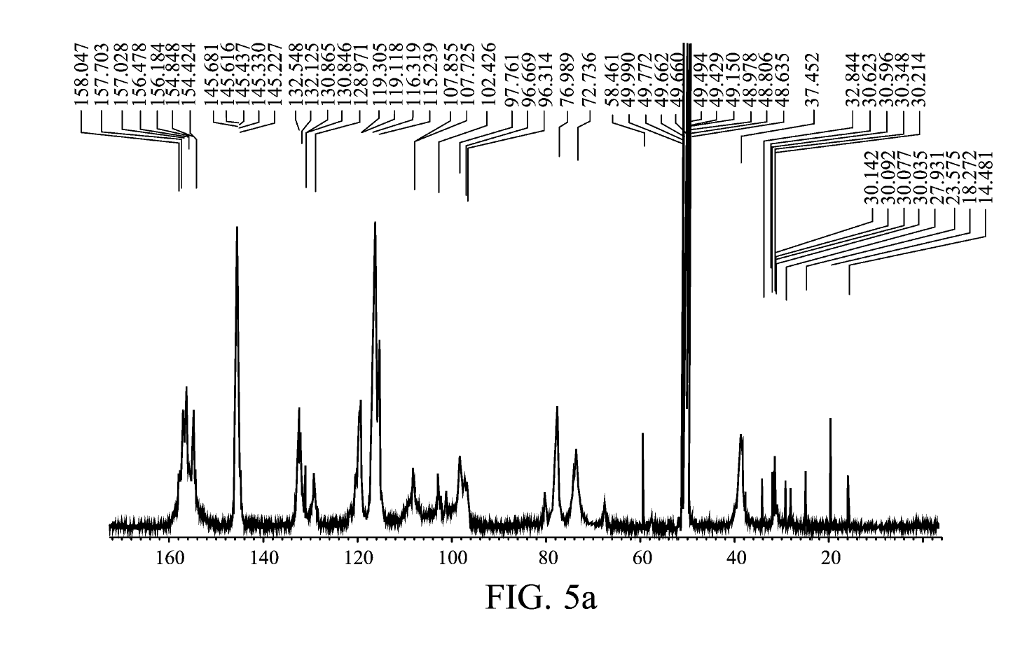

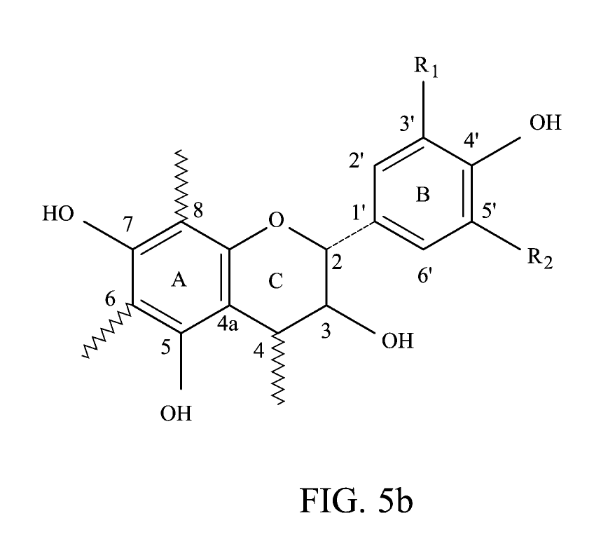

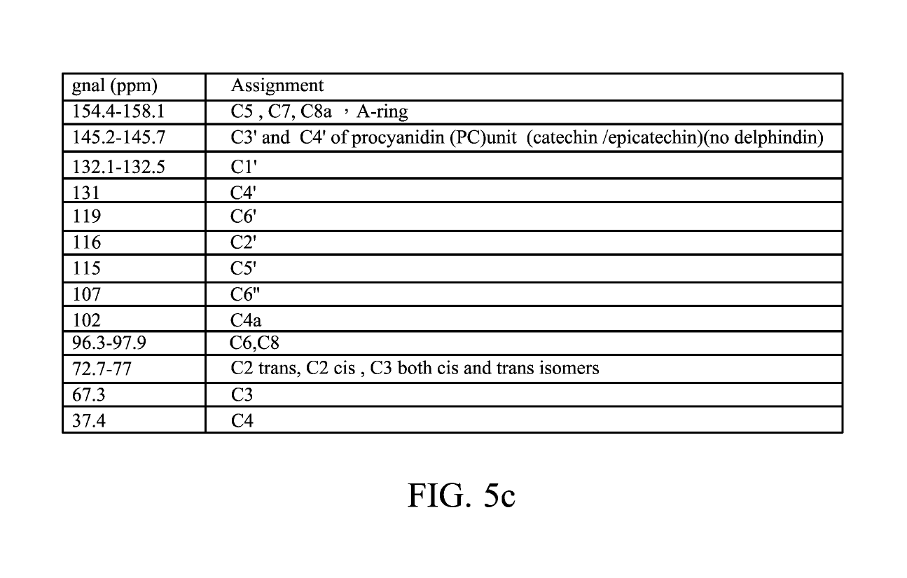

The purified proanthocyanidins sample was detected by ¹³C nuclear magnetic resonance spectrometry and ¹H nuclear magnetic resonance spectrometry. The ¹³C nuclear magnetic resonance spectroscopy results are shown as FIGS. 5a-5c, wherein at 145.2-145.7 ppm, there is only a peak of doublet-doublet and no other peak. Thus, the monomer had cyanidin but not delphindin, i.e. the B ring had three OH groups, which was identical with the EGA/MS analysis result. In FIG. 5b, R₁═H or OH and R₂═H, OH or OCH.

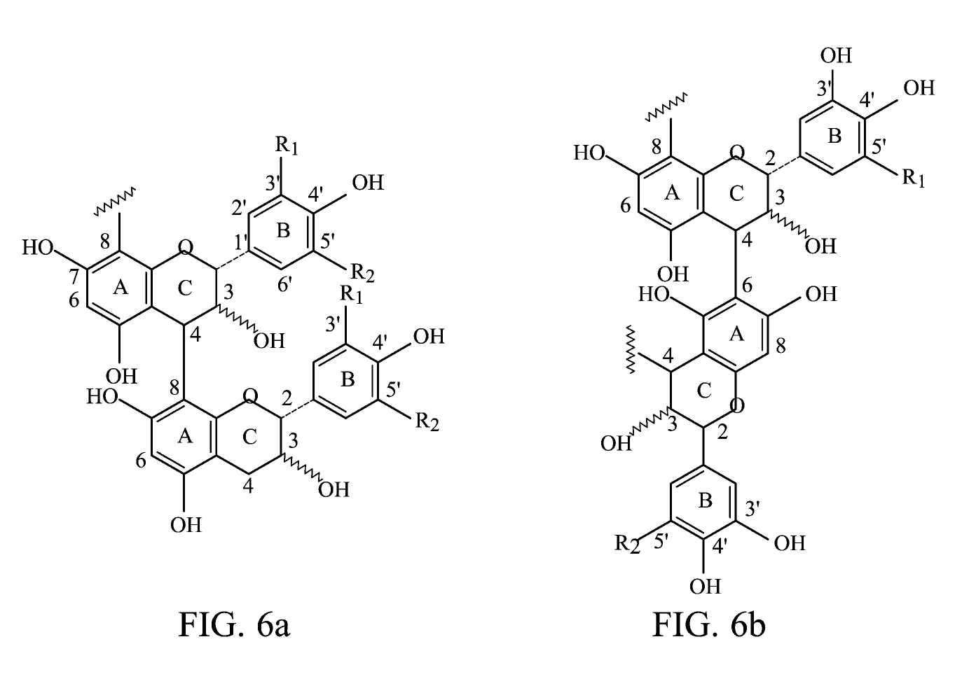

According the ¹³C nuclear magnetic resonance spectrum and the ¹H nuclear magnetic resonance spectrum, bonding between adjacent monomers of the proanthocyanidins mainly took place at the C4, C8 carbon-carbon bond. C4, C6 carbon-carbon bond unit and C2, C7 oxygen bond unit as shown in FIGS. 6a and 6b.

### Example 7

Matrix Assisted Laser Desorption Ionization Time-of-Flight (MALDI-TOF) Analysis

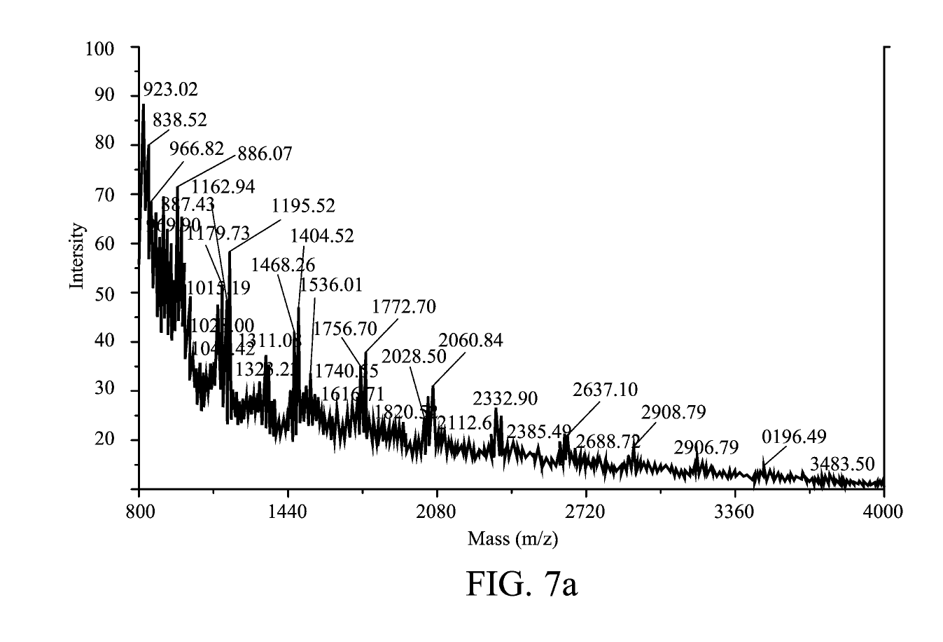

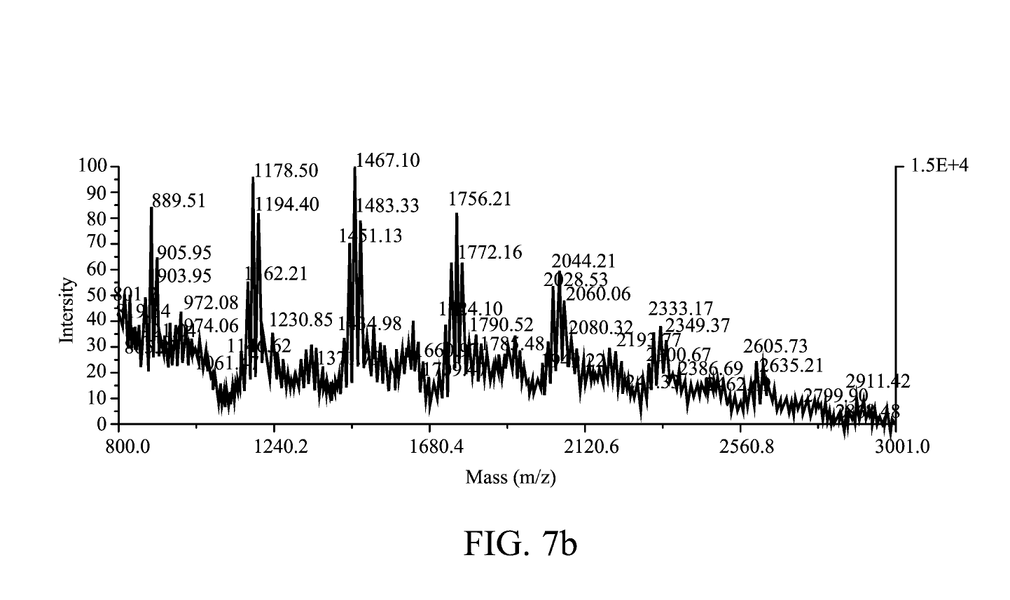

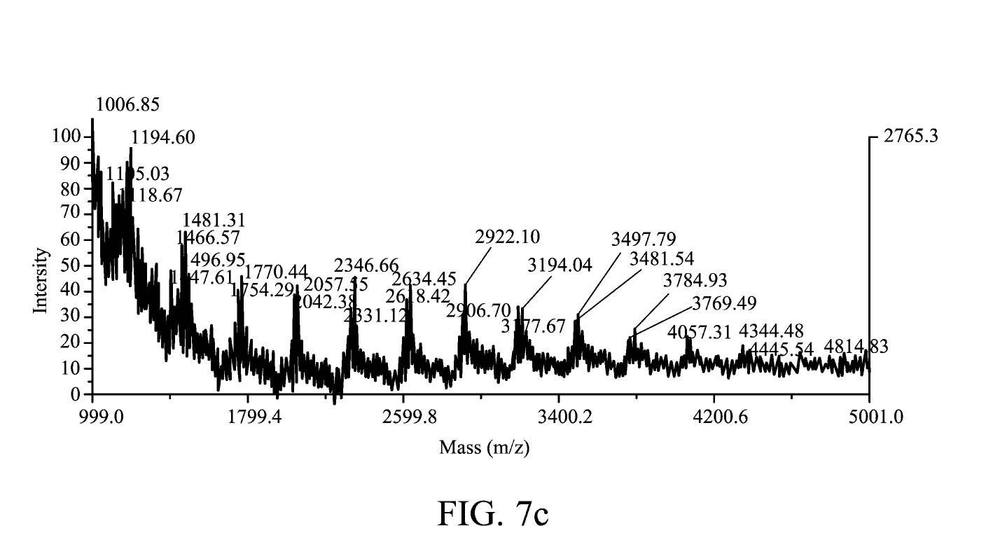

The molecular weight distribution of the partial purified proanthocyanidins was determined by matrix assisted laser desorption ionization time-of-flight mass spectrometry. The results were shown in FIGS. 7a-7c. The result showed that the molecular weight distribution of the partial purified proanthocyanidins was 500-5000. According the molecular weight distribution result, the polymerization degrees of the proanthocyanidins was presumed to be in the range of 2-18.

### Example 8

Inhibiting Hepatitis B Virus Activity Test for the Proanthocyanidins Extract from the Plants Belonging to Different Families

Plants belonging to different families were performed crude extractions. After being lyophilized, the extracts were analyzed for hepatitis B virus inhibiting effects thereof. By transfecting the plasmid with hepatitis B virus into cells, human hepatoma cell lines HepG 2.2.15 or 1.3ES8 producing hepatitis B virus was formed. The cells was added into 96 well cell cultured plates with a density of 1×10⁵ cells/100 μl/well and placed in a cell incubator for culturing over night. During the next day, the extract samples were prepared to have a concentration of 10 mg/ml with 10% DMSO and 90% sterilized water, and then diluted to a concentration suitable for testing with a cell cultured medium. The original medium in the 96 well cell cultured plates was removed without removing the cells and then the medium with the diluted sample was added into the 96 well cell cultured plates by a mount of 100 μl/well. Tests of inhibiting hepatitis B virus surface antigen (HBs) and hepatitis B virus e antigen (HBe) were conducting and after 4 days, the co-cultured mediums with diluted sample having no cytotoxicity (cell survival rate>85%) were selected to be analyzed. The hepatitis B virus surface antigen was detected by a SURASE B-96 (TMB) (GENERAL BIOLOGICALS) kit and hepatitis B virus e antigen was detected by an EASE BN-96 (TMB) (GENERAL BIOLOGICALS) kit. The culturing medium with diluted sample was added into the two 96 well cell cultured plates coated with anti hepatitis B virus surface antigen antibodies and coated with anti hepatitis B virus e antigen antibodies respectively, by a mount of 50 μl/well and 1000 μl/well, respectively and then placed at 40. The co-cultured medium with diluted sample in the 96 well cell cultured plates coated with anti hepatitis B virus surface antigen antibodies was left steady for 2 hours and then removed, while the co-cultured medium with diluted sample in the 96 well cell cultured plates coated with anti hepatitis B virus e antigen antibodies was placed over night and then removed. Then, the two 96 well cell cultured plate were washed 6 times with a washing buffer and added in secondary anti hepatitis B virus surface antigen antibodies conjugated with peroxidases and secondary anti hepatitis B virus e antigen antibodies conjugated with peroxidases, respectively, placed at 40 for 1 hour, and then the solution in the 96 well cell cultured plates was removed. The 96 well cell cultured plates were washed 6 times with a washing buffer. TBS was added into the 96 well cell cultured plates by a mount of 100 μl/well at room temperature in the dark for 30 minutes to perform chromatogenic reactions and then 2M sulfuric acid was added into the 96 well cell cultured plates by a mount of 100 μl/well to stop the chromatogenic reactions. After that, the two 96 well cell cultured plates were placed in an ELISA reader, respectively to be determine the absorbance at 450 nm. The absorbance of the control group without adding the co-cultured medium with diluted sample was used as a denominator and the difference between the absorbance of the control group and that of the experimental group was used as a numerator to calculate the hepatitis B virus inhibition rate of the extract sample.

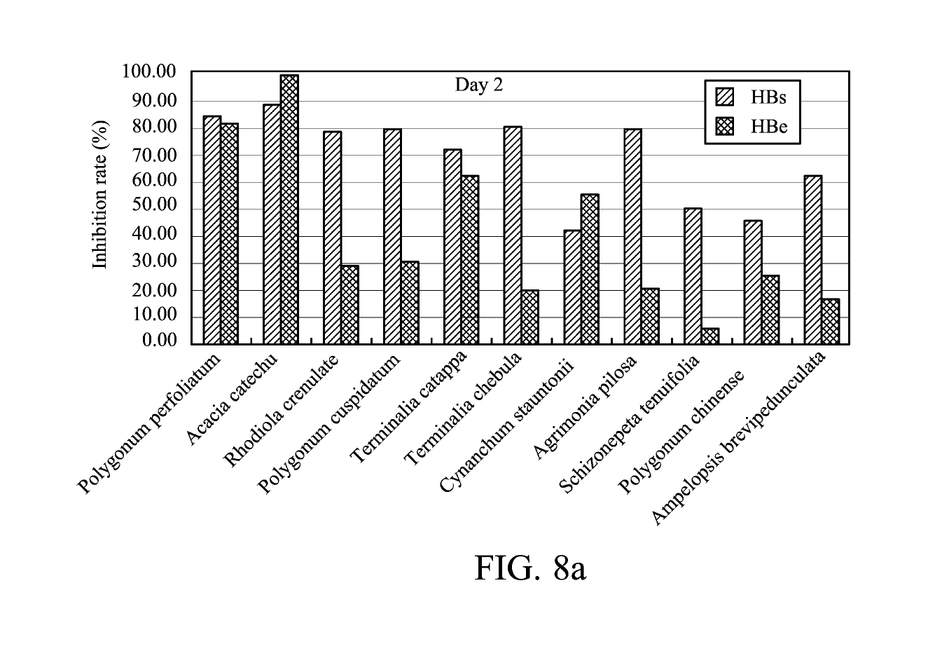

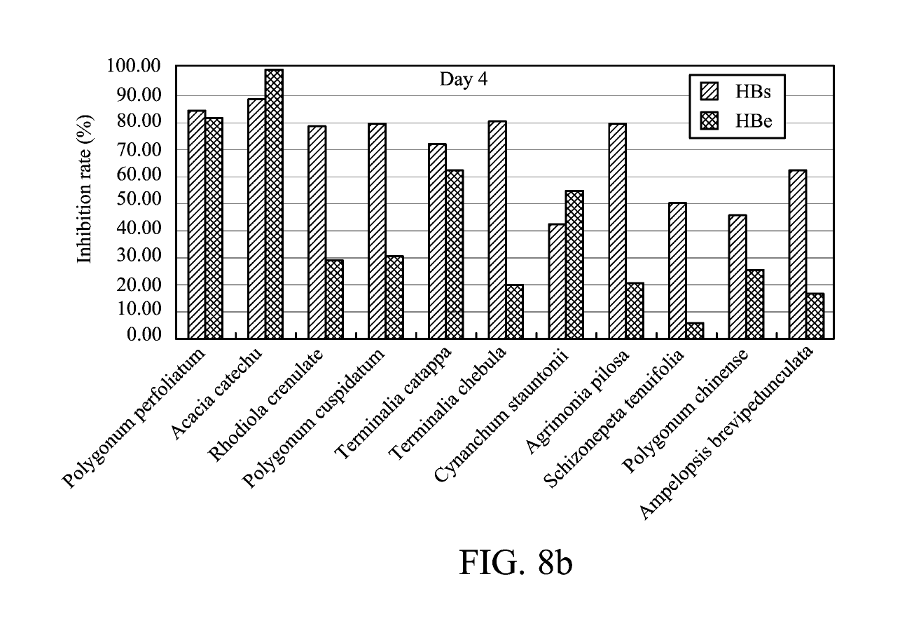

There were 11 proanthocyanidins containing plants extracted. The 11 proanthocyanidins containing plants comprised 1 plant belonging to the Leguminosae family, 1 plant belonging to the Crassulaceae family, 2 plants belonging to Combretaceae family, 1 plant belonging to the Asclepiadacea family, 1 plant belonging to the Rosaceae family, 1 plant belonging to the Lamiaceae family, 1 plant belonging to the Vitaceae family and 3 plants belonging to Polygonaceae family. The respective extracts of these plants were used to co-culture with cells with appropriate concentrations (74 g/ml or 25 g/ml) for 2 days and 4 days, respectively to determine the hepatitis B virus surface antigen and hepatitis B virus e antigen inhibiting abilities thereof. The results are shown in FIGS. 8a and 8b, respectively.

The results showed that the proanthocyanidins contained in the 11 plants mentioned above were able to inhibit the production of the hepatitis B virus antigen. Since the proanthocyanidins content in each plant was different, the inhibiting hepatitis B virus activity of each plant was different.

### Example 9

Inhibiting Hepatitis B Virus Activity Analysis for the Proanthocyanidins

7.0110 g of 95% ethanol extracted Boehmeria nivea (L.) Gaud sample was grinded, added into 300 ml of hexane and then heated and refluxed for 6 hours. Next, the solid part was taken (the liquid part was removed) and added into acetone/water (70/30) to be extracted. The water layer therefrom was taken and acetone was removed. The water layer was added into chloroform to extract the remains therein and then dried to obtain the sample 1. After that, 2.3116 g of the sample 1˜6 was placed into a gel permeation chromatography column and then continuously eluted with different solvents. The solvents were 300 ml of 95% ethanol, 300 ml of 95% ethanol/methanol (1/1. v/v), 300 ml of methanol, 300 ml of 50% methanol aqueous solution, 300 ml of 50% acetone aqueous solution and 300 ml of acetone, respectively. The samples 1-6 were performed the inhibiting hepatitis B virus activity test in Example 8. The culture medium with 50 μg/ml of CPB was used as a positive control while the culture medium without any extract therein was used as a negative control group. Each extract at each concentration was performed in the test for duplication.

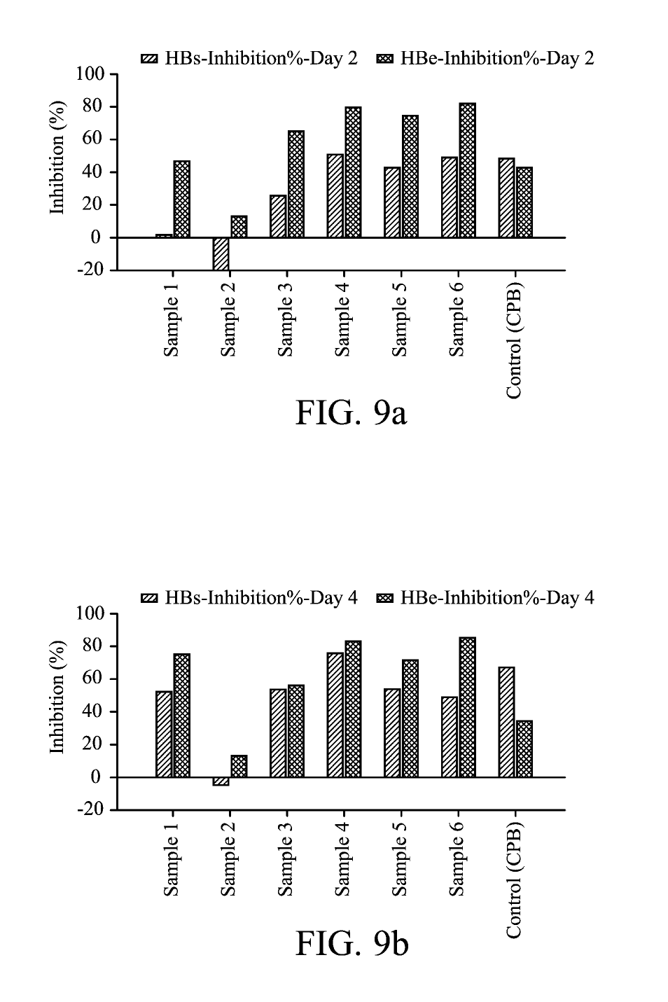

FIG. 9a shows that at day 2, all extracts except sample 2 inhibited the hepatitis B virus e antigen. FIG. 9b shows that at day 4, all extracts except sample 2 inhibited the hepatitis B virus surface antigen and hepatitis B virus e antigen.

### Example 10

Inhibiting Hepatitis B Virus Activity Analysis for Proanthocyanidins with Didderent Monomers Having Different R₁

Different samples A and B were used in the analyses in Examples 1-7 to identify the R₁ structures of the monomer of the proanthocyanidins between sample A and B and they were different. The 50% inhibition concentration (IC₅₀) test was performed on the two proanthocyanidins. Cells producing hepatitis B virus were co-cultured with the two samples with different concentrations. Then the inhibiting rate for hepatitis B virus surface antigen and hepatitis B virus e antigen of the two samples were determined. After serial dilution was performed on the samples and inhibition rate at different concentrations were determined, 50% inhibition concentration (IC₅₀) thereof was calculated by grafit5 software. The results are shown in the following table.

| | Day 2 | | Day 4 | |
|---|---|---|---|---|
| | IC₅₀ (μg/ml) | | IC₅₀ (μg/ml) | |
| | HBs | HBe | HBs | HBe |
| Sample A | 127.1 ± 13.4 | 84.1 ± 38.4 | 164.4 ± 36.7 | 47.4 ± 3.9 |
| Sample B | 158.8 ± 15.5 | 46.3 ± 11.2 | nd | 44.1 ± 6.3 |

Sample A: 99.3% of flavonols and 86.5% of proanthocyanidins
Sample B: proanthocyanidins which had been determined

The results showed that: at day 2, the 50% inhibition concentration (IC₅₀) of the sample A and sample B for hepatitis B virus surface antigen were 127.1±13.4 μg/ml and 158.8±15.5 μg/ml, respectively; at day 2, the 50% inhibition concentration (IC₅₀) of the sample A and sample B for hepatitis B virus e antigen were 84.1±38.4 μg/ml and 46.3±11.2 μg/ml, respectively; and at day 4, the 50% inhibition concentration (IC₅₀) of the sample A and sample B for hepatitis B virus e antigen were 47.4±3.9 μg/ml and 44.1±6.3 μg/ml, respectively. Thus, proanthocyanidins having different R₁ of the monomer have similar inhibiting hepatitis B virus bioactivity.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method for treating hepatitis B, comprising:
administering an effective amount of proanthocyanidins wherein the formula of a monomer of the proanthocyanidins is shown as Formula (I): 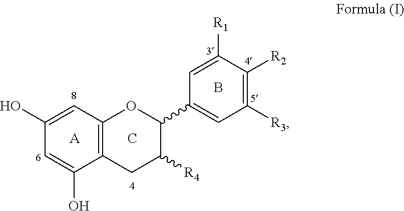
wherein R₁ is OCH₃, R₂ is OH and R₃ is H, or R₁ is OH and R₂ and R₃ both are H, or R₁ and R₂ both are OH and R₃ is H, or R₁, R₂ and R₃ all are OH, and R₄ is 3-(α)-OH, 3-(β)-OH, 3-(α)-O-sugar or 3-(β)-O-sugar.

2. The method for treating hepatitis B as claimed in claim 1, wherein a bonding between adjacent monomers of the proanthocyanidins take place at C4, C8 carbon-carbon bond, C4, C6 carbon-carbon bond or C2, C7 oxygen bond.

3. The method for treating hepatitis B as claimed in claim 1, wherein a polymerized number of the proanthocyanidins is about 2-30.

4. The method for treating hepatitis B as claimed in claim 1, wherein the monomer comprises (R) or (S) optical isomers at C4.

5. The method for treating hepatitis B as claimed in claim 1, wherein the monomer of the proanthocyanidins comprises a flavonoid.

6. The method for treating hepatitis B as claimed in claim 5, wherein the flavonoid comprises catechin, epicatechin, epiafzelechin, gallocatechin, galloepicatechin, epigallocatechin, flavonols, flavandiols, leucocyanidins or procynidins.

7. The method for treating hepatitis B as claimed in claim 1, wherein the monomer of the proanthocyanidins comprises a flavan-3-ol.

8. The method for treating hepatitis B as claimed in claim 1, wherein the proanthocyanidins is extracted from a plant.

9. The method for treating hepatitis B as claimed in claim 8, wherein the plant comprises a plant belonging to the Leguminosae, Crassulaceae, Combretaceae, Asclepiadaceae, Rosaceae, Lamiaceae, Polygonaceae, Ericaceae, Pinaceae, Vitaceae or Urticaceae family.

10. The method for treating hepatitis B as claimed in claim 9, wherein the plant belonging to the Urticaceae family comprises Boehmeria nivea (L.) Gaud.

11. The method for treating hepatitis B as claimed in claim 8, wherein the plant comprises Boehmeria nivea (L.) Gaud.
